SEQUENCE LISTING (5 sequences)

SEQ ID NO: 1 - DNA, 5440 bp, Mus musculus
ttgcacggtggagaaagtgagtcggcctctgtctgaggtcgggggtagggggcgggaccggggccaggccgatccggacccactggacacactgccctcttggagccggactctgcacccgacacgatgcgccggcctggcctcggcggcccctgccccctcttgctgctgctgctgctcccagccgccacatctgctagcggctccagtcccagccccagccccagccccatcgagaaagcggtggtcccgagccaccaggccggtgttgctgcctgtcactgctgcctagaccagacacctaagagtagccgctgcacccgagcttcctgcagggtccggaactgcccacctgccaagtgcacaggtctagaggggtgcctgaccccaacaccttcggtgcccagccccagccgcagtcccgtggagaagagtcaggtatccctcaactggcagccactgactttgcaggaggcccgagctctgctgaggcaaaggcggccccgagggccgtgggcccgggcattgctgaaaagaaggcccccacatcgcgcccctgccggccaggcccgggtcctatgtcccttgatctgtcacaacggtggtgtgtgtgtgaagcctgatcgctgcctttgccccccggacttcgctggcaaattctgccaattgcattcttcgggggcccggcccccggccccagccatgccgggccttacccgctccgtttatacaatgccactagccaaccaccgcgatgatgaacacggagtagcatcgatggtgagcgtgcacgtggagcaccctcaggaggcatcagtggtggtgcaccaagtggaacgtgtgtcgggcccctgggaggaggcgaaccctgaagccctagccagggctgaagcggcggcacgggccgaggcggcggcgccctacacagtgctggcacagagcgcgccacgcgaggacggctactcagacgcctccggcttcggttactgttttagagaactgcgcggaagcgaatgtgcatcgccgctgccagggctccggacgcaggaagtctgctgccgaggagagggcttggcctggggtgttcatgattgtcacccgtgcgcggaacacctgaggaactccaaccaagtgagtggcccaaatggaccttgtccacccggcttcgagagagttaatgggtcctgtgtagatgtggatgagtgcgccaccggtgggcgttgccaacacggggagtgtgccaacactcgtggcggctacacgtgtgtatgtccggacggtttcctcctggactcttcccgcagcagctgcatctcccaacacgtgatctccgaggcaaaggggccctgctaccgagtgctgcatgatggcggatgctcgctgcccattcttcgaaatatcaccaaacagatctgctgctgtagccgtgtgggcaaggcgtggggccgaggctgccagctctgtccaccttatggttcagagggttttcgggagatctgtccagctggccctggctaccactattcagcttctgacctccgatacaacaccagacccctaaaccaggatccacctcgagtgaccttcaatcagccacgtgttccaccagccacccctcggccacctacaggctttctgcctactcgtcgacctgagccccgccctgaccctgggccacagcctgaaccccggcctcggcctgaaccccggcctcggcctgaatcccgacctcggcctgaaccccggcctcggcctgaaccccggcctcagcctgaatcccagcctaggcctgaatcccggcctcggcctgaatcccagccttggcctgaatttcccctgcccagcatccctgcttggaccggtccagagattcctgaatcaggtccctcttccagcatgtgtcagcgaaatccccaggtttgtggtcctggacgctgcgttcctaggccaagcggctacacttgtgcgtgcgacccaggtttccggctcggcccccaggacactcgctgcattgacatagatgaatgtcgccgcgtccctacaccctgtgctcctgggcgttgcgagaatacaccaggcagctttcgctgcgtgtgcggcacgggcttccaagcaggcccgcgggctacagagtgcctggatgtggacgagtgccgccgcgtgccgccgccgtgtgaccgcgggcgctgcgagaacacgccaggcagtttcctatgtgtgtgtcccgccgggtaccaggcagcaccgcacggagccagctgccaggatgtggatgaatgcacccagagcccaggcctctgtggtcgcggggtctgcgaaaacctgcccggctctttccgctgtgtttgcccggctggcttccggggctcggcgtgtgaagaagatgtggatgaatgtgcccagcagcctccgccttgcgggccaggccgctgcgacaacaccgcgggttcgttccactgtgcctgcccagctggcttccgatcccgaggaccaggagccccctgccaagatgtggatgaatgttcccggagcccctcgccttgtgcctatggccgctgtgagaacacggaaggcagcttcaagtgtgtctgcccaacaggcttccaacccaacgctgcgggctccgagtgcgaggatgtggatgagtgtgagaaccgcttggcgtgtccagggcaggagtgtgtgaactcaccgggctccttccagtgccgggcctgccctgttgggcaccacctgcaccgtggcagatgtactgatgtggatgaatgcagttcgggcaccccttgtggtctccatggccagtgcacaaatactaaaggctccttccactgcagctgctcgacaggttaccgggcgccatctggtcaacctgggccttgtgcagacataaatgagtgtctggaaggcgacttctgcttcccccacggagagtgcctcaacactgatggctcctttacctgtacctgtgcccctggctaccggcctggacctcgtggagcatcttgtctggacgtggacgagtgcagcgaagaggatctttgccagagcggcatctgtaccaacactgacggctccttcgagtgcatctgtcctcctggacaccgcgctggccctgaccttgcctcctgccttgacattgacgagtgtcgtgaacggggacctgccctgtgcgggtctcagcgctgtgaaaattctcctggttcctaccgctgtgtgcgggactgcgatccaggttaccaccccggccctgagggcacctgtgatgacattgatgagtgccgagaatacggctctgcgatttgtggtgcccagcgctgtgagaacaccccgggctcctaccgctgcacaccagcctgcgaccctggctatcagcctacaccagggggcgggtgccaagatgtggatgaatgccggaaccggtctttctgcggcgcccatgctatgtgccagaatctgcctgggtccttccagtgcgtctgtgaccaaggctacgaaggggcacgggacggacgtcactgcgtggatgtgaatgagtgtgagacactacagggcgtgtgtggctccgcgctgtgtgaaaatgttgaaggctccttcctgtgtgtctgccccaatagccctgaggaatttgatcccatgactggacgctgtgttcccccacgggctcctgcaggcacgttccctggctcccagccccaagcacctgccagccccagtcttccagccaggccaccagcccctcccccacctcgaagacccagccctcccaggcagggtcccgtgagcagtggacgtcgcgagtgctactttgatacagccgctccagatgcgtgtgacaatatcttggcccgaaacgtgacgtggcaggagtgctgctgtactgtgggagaaggctggggcagcggctgccgcatccaacagtgcccgggcaccgagacagctgaataccagtcattgtgtcctcacggccggggctacctggtgcccagtggagacctgagtgcccggagagatgtggacgaatgccaactcttccaggaccaggtgtgcaagagcggagtgtgtgtgaacacggccccaggctactcttgctattgcagcaacgggttctactatcatgcacacaggctggagtgcgttgataatgacgaatgtgcggacgaggagcctgcttgtgaaggtggccgctgcgtcaacacggtgggctcttaccactgtacctgcgagcccccactggtgctggacggctcccggcgacgctgcgtctccaacgagagccagagcctggatgacaatctaggagtgtgctggcaggaagtgggacctgatctcgtctgcagtcgcccacgactagaccggcaggccacctacaccgagtgctgctgtctctatggtgaagcctggggtatggactgcgctctctgtcctgcccaggactcagatgactttgaggccctttgcaatgtgctgcgtccacctgcctatggcccgccgcgccctggtggctttggaattccctatgaatatggcccagatataggcccaccttatcagagtctcccctatgggccagatctgtatccaccgccagtgctaccctacgacccctacccacccccacctggaccctttgctcgccgggaggccccttatggcgccccgcccttcgacatgccggactttgaggatgatggtggcccctatggcgagtctgagactcctgacccacctagccgaggcactggttggccttatcggtccagagacacccgtggctccttcccagagcctgaggaatcctctgagcgtggaagctatacaggagccttgtctgagccctatgagggcctagaggccgaagagtgtggtatcctggatggctgccctcacggccgctgtgtacgcgttccagaaggcttcacctgcgattgtttcgatggctaccgcctggacatcacccgcatgtcctgtgtcgacgtcaacgagtgtgatgaggcagaggcaacctccccactctgcatcaatgcgcgctgtgtcaacactgacggctccttccgctgtatctgtcgtccaggatttgctcccacgcatcagccacatcactgtgcgcctgctcgaccccgggcctgagcaggggtgccaaatctcccactatacctgagcaccgggagcttccagagcacgcctgcctccctccctctagtctgcatgtgtgcaaggatgtcagacagtctcgacttaggatggacagacacagaggggtgccttctatggtgccagcctgtcatgcccagtccctcccatcagtgccctgggcctgtcctgggtcccctgacacattacttgcccttttatacaattttcaattaaaacacctattttgtaaaaaaaaaaaaaaaaaaaaaaaaaaaaaaaaaaaaaaaaaaaaaaaaaaaaaaaaaaa

SEQ ID NO: 2 - DNA, 5120 bp, Mus musculus
tggagcgcggcgctgcagccatggcgggcggcgcgcagctgctctgggtgtcgctattggtgctgctggcgcagctggggccgcagcccgtactgggccgaccccgagagcgtcttcgcgtgcgcttcacccccgctgtgtgcggcctgcgttgcatccacgggcccactggctcccgctgtaccccgacctgcgcgccccgcaacgccaccagcgtggacagcggcgcgcccggcggggcggccccggggggacccggcttccgcgccttcctatgtcccttgatctgtcacaacggtggtgtgtgtgtgaagcctgatcgctgcctttgccccccggacttcgctggcaaattctgccaattgcattcttcgggggcccggcccccggccccagccatgccgggccttacccgctccgtttatacaatgccactagccaaccaccgcgatgatgaacacggagtagcatcgatggtgagcgtgcacgtggagcaccctcaggaggcatcagtggtggtgcaccaagtggaacgtgtgtcgggcccctgggaggaggcgaaccctgaagccctagccagggctgaagcggcggcacgggccgaggcggcggcgccctacacagtgctggcacagagcgcgccacgcgaggacggctactcagacgcctccggcttcggttactgttttagagaactgcgcggaagcgaatgtgcatcgccgctgccagggctccggacgcaggaagtctgctgccgaggagagggcttggcctggggtgttcatgattgtcacccgtgcgcggaacacctgaggaactccaaccaagtgagtggcccaaatggaccttgtccacccggcttcgagagagttaatgggtcctgtgtagatgtggatgagtgcgccaccggtgggcgttgccaacacggggagtgtgccaacactcgtggcggctacacgtgtgtatgtccggacggtttcctcctggactcttcccgcagcagctgcatctcccaacacgtgatctccgaggcaaaggggccctgctaccgagtgctgcatgatggcggatgctcgctgcccattcttcgaaatatcaccaaacagatctgctgctgtagccgtgtgggcaaggcgtggggccgaggctgccagctctgtccaccttatggttcagagggttttcgggagatctgtccagctggccctggctaccactattcagcttctgacctccgatacaacaccagacccctaaaccaggatccacctcgagtgaccttcaatcagccacgtgttccaccagccacccctcggccacctacaggctttctgcctactcgtcgacctgagccccgccctgaccctgggccacagcctgaaccccggcctcggcctgaaccccggcctcggcctgaatcccgacctcggcctgaaccccggcctcggcctgaaccccggcctcagcctgaatcccagcctaggcctgaatcccggcctcggcctgaatcccagccttggcctgaatttcccctgcccagcatccctgcttggaccggtccagagattcctgaatcaggtccctcttccagcatgtgtcagcgaaatccccaggtttgtggtcctggacgctgcgttcctaggccaagcggctacacttgtgcgtgcgacccaggtttccggctcggcccccagggcactcgctgcattgacatagatgaatgtcgccgcgtccctacaccctgtgctcctgggcgttgcgagaatacaccaggcagctttcgctgcgtgtgcggcacgggcttccaagcaggcccgcgggctacagagtgcctggatgtggacgagtgccgccgcgtgccgccgccgtgtgaccgcgggcgctgcgagaacacgccaggcagtttcctatgtgtgtgtcccgccgggtaccaggcagcaccgcacggagccagctgccaggatgtggatgaatgcacccagagcccaggcctctgtggtcgcggggtctgcgaaaacctgcccggctctttccgctgtgtttgcccggctggcttccggggctcggcgtgtgaagaagatgtggatgaatgtgcccagcagcctccgccttgcgggccaggccgctgcgacaacaccgcgggttcgttccactgtgcctgcccagctggcttccgatcccgaggaccaggagccccctgccaagatgtggatgaatgttcccggagcccctcgccttgtgcctatggccgctgtgagaacacggaaggcagcttcaagtgtgtctgcccaacaggcttccaacccaacgctgcgggctccgagtgcgaggatgtggatgagtgtgagaaccgcttggcgtgtccagggcaggagtgtgtgaactcaccgggctccttccagtgccgggcctgccctgttgggcaccacctgcaccgtggcagatgtactgatgtggatgaatgcagttcgggcaccccttgtggtctccatggccagtgcacaaatactaaaggctccttccactgcagctgctcgacaggttaccgggcgccatctggtcaacctgggccttgtgcagacataaatgagtgtctggaaggcgacttctgcttcccccacggagagtgcctcaacactgatggctcctttacctgtacctgtgcccctggctaccggcctggacctcgtggagcatcttgtctggacgtggacgagtgcagcgaagaggatctttgccagagcggcatctgtaccaacactgacggctccttcgagtgcatctgtcctcctggacaccgcgctggccctgaccttgcctcctgccttgacattgacgagtgtcgtgaacggggacctgccctgtgcgggtctcagcgctgtgaaaattctcctggttcctaccgctgtgtgcgggactgcgatccaggttaccaccccggccctgagggcacctgtgatgacattgatgagtgccgagaatacggctctgcgatttgtggtgcccagcgctgtgagaacaccccgggctcctaccgctgcacaccagcctgcgaccctggctatcagcctacaccagggggcgggtgccaagatgtggatgaatgccggaaccggtctttctgcggcgcccatgctatgtgccagaatctgcctgggtccttccagtgcgtctgtgaccaaggctacgaaggggcacgggacggacgtcactgcgtggatgtgaatgagtgtgagacactacagggcgtgtgtggctccgcgctgtgtgaaaatgttgaaggctccttcctgtgtgtctgccccaatagccctgaggaatttgatcccatgactggacgctgtgttcccccacgggctcctgcaggcacgttccctggctcccagccccaagcacctgccagccccagtcttccagccaggccaccagcccctcccccacctcgaagacccagccctcccaggcagggtcccgtgagcagtggacgtcgcgagtgctactttgatacagccgctccagatgcgtgtgacaatatcttggcccgaaacgtgacgtggcaggagtgctgctgtactgtgggagaaggctggggcagcggctgccgcatccaacagtgcccgggcaccgagacagctgaataccagtcattgtgtcctcacggccggggctacctggtgcccagtggagacctgagtgcccggagagatgtggacgaatgccaactcttccaggaccaggtgtgcaagagcggagtgtgtgtgaacacggccccaggctactcttgctattgcagcaacgggttctactatcatgcacacaggctggagtgcgttgataatgacgaatgtgcggacgaggagcctgcttgtgaaggtggccgctgcgtcaacacggtgggctcttaccactgtacctgcgagcccccactggtgctggacggctcccggcgacgctgcgtctccaacgagagccagagcctggatgacaatctaggagtgtgctggcaggaagtgggacctgatctcgtctgcagtcgcccacgactagaccggcaggccacctacaccgagtgctgctgtctctatggtgaagcctggggtatggactgcgctctctgtcctgcccaggactcagatgactttgaggccctttgcaatgtgctgcgtccacctgcctatggcccgccgcgccctggtggctttggaattccctatgaatatggcccagatataggcccaccttatcagagtctcccctatgggccagatctgtatccaccgccagtgctaccctacgacccctacccacccccacctggaccctttgctcgccgggaggccccttatggcgccccgcccttcgacatgccggactttgaggatgatggtggcccctatggcgagtctgagactcctgacccacctagccgaggcactggttggccttatcggtccagagacacccgtggctccttcccagagcctgaggaatcctctgagcgtggaagctatacaggagccttgtctgagccctatgagggcctagaggccgaagagtgtggtatcctggatggctgccctcacggccgctgtgtacgcgttccagaaggcttcacctgcgattgtttcgatggctaccgcctggacatcacccgcatgtcctgtgtcgacgtcaacgagtgtgatgaggcagaggcaacctccccactctgcatcaatgcgcgctgtgtcaacactgacggctccttccgctgtatctgtcgtccaggatttgctcccacgcatcagccacatcactgtgcgcctgctcgaccccgggcctgagcaggggtgccaaatctcccactatacctgagcaccgggagcttccagagcacgcctgcctccctccctctagtctgcatgtgtgcaaggatgtcagacagtctcgacttaggatggacagacacagaggggtgccttctatggtgccagcctgtcatgcccagtccctcccatcagtgccctgggcctgtcctgggtcccctgacacattacttgcccttttatacaattttcaattaaaacacctattttgtaaaaaaaaaaaaaaaaaaaaaaaaaaaaaaaaaaaaaaaaaaa

SEQ ID NO: 3 - DNA, 24 bp, Artificial Sequence
  Primer
aagagggagaagaaactgggctgg

SEQ ID NO: 4 - DNA, 20 bp, Artificial Sequence
  Primer
tcctgtagtgtttgcccctg

SEQ ID NO: 5 - DNA, 3036 bp, Mus musculus
gtgagtgcgggatggtgctcccaagggaggagtgcccggagggaggaccctggagacaatgccttagggagcaggagcccagattccatgtacagaaatgctggggactccgtggggtctttgaggaagggggctctgaattccttatttggggagtggatctccagggcgctgatttcaggagcattgagtgtttttggaactgtggtgtcccacgtgaacggactgagactagctttcatccctttcagtcaaggcgaggatcttaggatctgggaacttttgagatggggcccaaattcgcagactgtgcgtagtgggtcagcaaggagtgtttcagagtcgcatggggtggtggggcatatctcaagtttctaggtctgtctttgagtgggggaagactaggaaagtctggcatgggactgcttgtgatgaagagctaagcgggaggggacccaggaaagacggggtgcctttcggcaagacctgagttacagcggttccacgtgcgacagatatccagataggaggcctgaggaaagggcgccccctgggggaggaagggaccggcttgcgcttcagcttcctctagacaaggggagcattctggggttcaggtgaggtcaggagggtgcgccacctgctagcccagagtgtggggatggacttgggggtcgggttttcattgcttctcaccccttacgccctccggaatggggagtggggcccctggcgccgagaactcgcccaggctggcccccagccagcccaacccggccggcgtgagctcatctcccgcccgagcccccgcccgctaccactccctctctccgccccttcctccctccttccttcactctctcccggcccagccccaactcgcccgtgccaggcgccgtgcccagacgggtgccaaacccctggaggcacaaggaggaacatgtgaaggcgggctgacctcggcaagacccctatgtccttagcttcctgttcttccaatgttgtgggacatgaagttagttttgcccatttagcagcaagccctctgatatgggcaccctgagctgggagtctgtattcaaggcaagggcaggcagtgaaatgacttaagagactaagaaatgcccctaaacgtggtggcttcctcccatctgagacacttccatttttccaatcttgcttctttgctgagcccttcttaagctctgcccttaaaaaaaaaaaaaaaattaaggtacctggagtccaggtttaacgctcctgacaggaaatccgggtttttggatcccggatccaaaggtccctcttcacccaggggcaaacactacaggaactcctccagcccagtttcttctccctctttggaggtactcctccaagctgcgcccctcccaggagaattctaatattgtcacccagcggcttctcgcttcccctgaaacattcaagcatgagccttaagtggagatacttctccccctccaacatctcccccgattctcttcccacgtagcttcatttttccttctctttctaaggtagccttcccagacctatccacacagggccattaatcaagactcggggcaaccagatccttggctagggaggagtaggactttccagcgattcctaaggtccccgcaacttgagagttttaacgtgctagaaacgatcagactccacttctccttgtcctcagctccctcacatgggccacacccccgcctccagaaagtactagtcctgttttctaggcaactctccgaaaggacttgaaagactcttggttgagagcctagatttgttctgagctagcctcaccctgcctcctagaaaacttgggttttctttgaacctcagagtactgcctccatgctcatatcttccaaagacgcctgtcccttgattcgtctagactgaggtggctcccctctgcctccttttgtatggctgtggctcagaaaatcacacacacacacacacacacacctcccaggacagcccgggctgtctgctccctccttcccctgagtgcagctgtctccagctgttttcggcagccccatgggaggaagggaggggggtgccctcgaaagacacgtccggcctcctttggctcagcagtccggggctccccaagggtccggaatgcggagacgacgccctgcctccgctgcacccctttctttgtttaccccaccgcacagctggcagtgccccgcccagaggtggccaagggcccagagagggcgcgcggaggggagtagaggcgcgtccccgctagttgccccgcccgatgctaccggaggccgggagggaaggctgaaaagctggctgtgggcggggctaaggcgcctagctcgcccaagagtgggaggggcgtgattcggagcaaatcagagggaggaaagtgagtaggcggggctaaggctaaggtacctgggttaggcggagtttacaatgtcaggtagaaggccatgtgggcgggactagaaaggggtgcctagccaatgggaaaagcctgaactttttggattccagctgagacacaaaagggaaacttcggtttgttttgagcgaaggtaatccagtgggcgggaatatcactgtttgggcggggctgggacgcccgaacttaaacagaaaatgggaaggagaatgaaatagagggaggaggctgagtaaacccacctaaaaggcgggactatgtgatagtcgcgcgacttgacccacccgacatagtcagccagatttggggaaggttgttgtggtagcatactgctataatcccaaaaattgggaagcggagtcaggagtcaagagttaaagcattggctatatagtgagttttcgagactagcctgggctacattagtccctatcttaaaacaaaagcaaacaaacgggaaaagtcacctacgatggagagatgtaaggatggtgtaccttgaggccctcgtttcagtgctgggtactgagttagctgtctgccaggcacagtactgatattttgtatccttgctttcttctcag

(19) United States
(12) Patent Application Publication
(10) Pub. No.: US 2010/0077490 A1
(43) Pub. Date: Mar. 25, 2010

(54) Animal model exhibiting cancer, pulmonary emphysema and cardiomyopathy

(75) Inventors: Harald von Melchner, Kronberg/Taunus (DE); Imgard S. Thorey, Zurich (CH); Frank Wempe, Muhlheim am Main (DE); Anja Stemer-Kock, Morfelden-Walldorf (DE); Jorma Keski-Oja, Helsinki (FI)

Correspondence Address: Olson & Cepuritis, LTD., 20 NORTH WACKER DRIVE, 36TH FLOOR, CHICAGO, IL, 60606, US

(73) Assignee: FrankGen Biotechnologie AG

(21) Appl. No.: 12/321,839

(22) Filed: Jan. 26, 2009

Related U.S. Application Data
(63) Continuation of application No. 10/485,891, filed on Jul. 22, 2004, now abandoned.
(60) Provisional application No. 60/312,164, filed on Aug. 14, 2001.

(51) Int. Cl. G01N 33/574; A01K 67/027; G01N 33/50; C12N 5/07

(52) U.S. Cl. 800/3; 800/10; 800/9; 435/354

(57) ABSTRACT

A transgenic mouse model for colorectal cancer, pulmonary emphysema or cardiomyopathy comprises a transgenic mouse that produces suboptimal levels of latent transforming growth factor β binding protein 4 (LTBP-4). The transgenic mouse includes a mutation resulting in homozygous disruption of both endogenous alleles of a gene encoding LTBP-4, and develops colorectal cancer, pulmonary emphysema or cardiomyopathy.

This application is a continuation of U.S. Ser. No. 10/485,891, filed on Jul. 22, 2004, which is the National Stage of International Application No. PCT/EP02/09011 filed on Aug. 12, 2002, which claims the benefit of U.S. Provisional Application for Patent Ser. No. 60/312,164, filed Aug. 14, 2001; each of which is incorporated herein by reference.

The present invention relates to an animal model and cells which do not produce functional latent transforming growth factor β binding protein 4 or which produce suboptimal levels of latent transforming growth factor β binding protein 4. Furthermore, the present invention relates to methods and kits for diagnosing cancer, pulmonary emphysema or cardiomyopathy and analyzing whether cancer and/or pulmonary emphysema and/or cardiomyopathy are caused by a differential expression of LTBP-4 or by a defect in LTBP-4 gene. Moreover the present invention relates to selecting an agent for treating a symptom occurring in the animal model of the invention.

### BACKGROUND OF THE INVENTION

Transforming growth factor-β (TGF-β) belongs to a protein superfamily whose members are necessary for normal development. They also control cell growth and differentiation in a variety of adult tissues and are involved in a wide range of immune reactions (reviewed in Massague, J., et al. (2000) Cell 103, 295-309, Massague, J. & Wotton, D. (2000) Embo J 19, 1745-1754). Most cells secrete TGF-13 as a functionally inactive (latent) precursor cytokine that interacts with its receptors only after proteolytic activation. There are two known, structurally different latent complexes of TGF-β. A small latent complex consists of a mature TGF-13 dimer bound to the N-terminal portion of the TGF-β precursor, i.e. the latency associated peptide (LAP). The large latent complex contains, in addition to TGF-β-LAP, the latent TGF-β binding protein (LTBP). Unless proteolytically cleaved from LTBPs after secretion, TGF-β is deposited in the extracellular matrix (ECM) as a high molecular weight complex with LTBP and LAP (reviewed in Koli, K., et al. (2001) Microsc Res Tech 52, 354-362, Taipale, J., et al. (1998) Adv Cancer Res 75, 87-134).

LTBPs are glycoproteins that belong to the family of fibrillins. More than 60% of the protein structure of the LTBP family is composed of repetitive EGF like domains and 8-Cystein repeats, both of which are believed to mediate protein-protein interactions. Like fibrillins, LTBPs are structural components of the extracellular matrix (ECM) and bind to elastic fibers. Although LTBPs are not directly involved in TGF-β latency most cells secrete TGF-β in complex with LTBP and, by binding TGF-β to the ECM, form extracellular deposits of TGF-β. This is believed to provide tissues with a rapidly inducible and highly localized TGF-β signal (Koli, K. et al. (2001); Microsc Res Tech 52, 354-362). Four isoforms of LTBPs (LTBP 1-4) have been cloned thus far (Tsuji, T. et al. (1990); Proc Natl Acad Sci USA 87, 8835-8839, Moren, A. et al. (1994); J Biol Chem 269, 32469-32478, Yin, W. et al. (1995) J Biol Chem 270, 10147-10160, Giltay, R., et al. (1997); FEBS Lett 411, 164-168, Saharinen, J. et al. (1998); J Biol Chem 273, 18459-18469). Each has several splice variants and at least one (LTBP1) has two alternative promoters (Koski, C., et al. (1999); J Biol Chem 274, 32619-32630). The expression of both, isoforms and splice variants, appears tightly controlled in a tissue specific manner. The function of LTBPs at organismal level is largely unknown.

As it is evident from the prior art there is a need for animal models to elucidate the function of LTBPs and in particular LTBP-4. A further object is to provide cells lacking LTBP-4 protein.

As a solution to this object a non-human animal model and cells which do not produce functional latent transforming growth factor β binding protein 4 (LTBP-4) or which produce suboptimal levels of latent transforming growth factor β binding protein 4 (LTBP-4) were provided.

### DESCRIPTION OF THE INVENTION

The present invention relates to a non-human animal model which does not produce functional latent transforming growth factor β binding protein 4 (LTBP-4) or which produces suboptimal levels of latent transforming growth factor β binding protein 4 (LTBP-4).

In a preferred embodiment the genome of the animal comprises a homozygous disruption of the LTBP-4 gene. Preferably this homozygous disruption has been generated by a mutation and this mutation can be an insertion, deletion or a substitution mutation. Furthermore, preferably said mutation is generated by gene targeting, gene trap integration or mutagenesis and it has occurred in an exon, intron, regulator region or splicing site of the LTBP-4 gene, preferably in intron 5 of the LTBP-4 gene.

In a further preferred embodiment the animal model exhibits cancer (e.g. a colon cancer), pulmonary emphysema or cardiomyopathy. Furthermore, the animal is preferably a mammal, more preferably a mouse or a rat.

In a further aspect the invention relates to a cell isolated from a non-human animal model whose genome comprises a homozygous disruption of the LTBP-4 gene such that said gene does not produce functional latent transforming growth factor β binding protein 4 or such that said gene produces suboptimal levels of latent transforming growth factor β binding protein 4. Preferably, the cell is from a colon, lung or heart.

In a further aspect the invention is directed to a cell whose genome comprises a homozygous disruption of the LTBP-4 gene. The cell is, preferably, an embryonic stem cell.

In a further embodiment the present invention is directed to a method for selecting an agent for treating a symptom occurring in the animal model of the invention comprising: a) applying one or more agents to be tested to the animal model of the invention; and b) determining whether one or more symptoms occurring in the animal model of the present invention have changed as a result of application of said agent or agents. In a preferred embodiment the symptom is selected from a group consisting of cancer, pulmonary emphysema and cardiomyopathy.

In a further aspect of the invention the agent which is suitable for treating a symptom occurring in the animal model of the invention is a pharmaceutical. Furthermore, the present invention relates to the use of the agent which is suitable for treating a symptom occurring in the animal model of the invention for the preparation of a pharmaceutical composition for the treatment of cancer, pulmonary emphysema or cardiomyopathy. Moreover, the present invention is directed to a method of treatment of cancer, pulmonary emphysema or cardiomyopathy using the agent which is suitable for treating a symptom occurring in the animal model of the present invention.

A further aspect of the invention is a method to analyze whether cancer, pulmonary emphysema or cardiomyopathy is caused by differential LTBP-4 gene or protein expression or expression level or by a defect in the LTBP-4 gene comprising:

a) characterizing the LTBP-4 gene or protein expression or expression level or LTBP-4 gene allele status of an individual having cancer and/or pulmonary emphysema and/or cardiomyopathy, and b) characterizing the LTBP-4 gene or protein expression or expression level or LTBP-4 gene allele status of a control, a difference in the LTBP-4 gene or protein expression or expression level or LTBP-4 gene allele status indicating that cancer and/or pulmonary emphysema and/or cardiomyopathy is caused by differential LTBP-4 gene or protein expression or expression level or by a defect in the LTBP-4 gene.

Furthermore, one aspect of the invention relates to a method for diagnosing cancer, pulmonary emphysema or cardiomyopathy comprising: a) characterizing the LTBP-4 gene or protein expression or expression level or LTBP-4 gene allele status of an individual, b) characterizing the LTBP-4 gene or protein expression or expression level or LTBP-4 gene allele status of a control, a difference in the LTBP-4 gene or protein expression or expression level or LTBP-4 gene allele status indicating the presence of cancer and/or pulmonary emphysema and/or cardiomyopathy in said individual.

In a preferred embodiment of the methods to analyze whether cancer and/or pulmonary emphysema and/or cardiomyopathy is caused by LTBP-4 and for diagnosing cancer, pulmonary emphysema or cardiomyopathy, the LTBP-4 gene and the LTBP-4 expression or expression level is detected by RT-PCR, Northern analysis, microarray (DNA chip) analysis or antibodies directed to LTBP-4 protein and the LTBP-4 gene allele status is detected by mutation screening.

A further aspect of the invention relates to a kit for diagnosing cancer and/or pulmonary emphysema and/or cardiomyopathy comprising: a) means to detect the LTBP-4 gene or protein expression or expression level or LTBP-4 gene allele status of an individual and control; and b) instructions for detecting the LTBP-4 gene or protein expression or expression level or LTBP-4 gene allele status.

The term “does not produce functional latent transforming growth factor β binding protein 4 (LTBP-4)” denotes the lack of translation of an effective LTBP-4 protein.

The term “produces suboptimal levels of latent transforming growth factor β binding protein 4 (LTBP-4)” encompasses the translation level of LTBP-4 protein, which is insufficient to produce an effective concentration of LTBP-4. Preferably, the level is reduced at least by 50%, more preferably by 70% and most preferably by 95%.

The term “homozygous disruption of the LTBP-4 gene” relates to an identical mutation in both LTBP-4 alleles.

The term “mutation” refers to a change of one or more nucleotide pairs of a DNA molecule.

The term “insertion” is directed to a mutation identified by the presence of one or more additional base pairs in the DNA. The term “deletion” relates to a mutation generated by removal of a sequence of DNA (one or more base pairs), the regions on either side being joined together. The term “substitution mutation” is directed to a nucleotide change. The substitution mutation can result in an amino acid change or can introduce a premature translation stop codon. Furthermore, a substitution mutation can affect splicing or expression of the gene when occurring in the sites necessary for splicing or regulatory regions of the gene.

The term “gene targeting” relates to a type of homologous recombination that occurs when a fragment of genomic DNA is introduced into a cell and that fragment locates and recombinates with homologous sequences. The term “gene trap integration” is directed to insertion of a vector, which comprises a reporter gene and which is activated upon its insertion within an active transcription unit, into a chromosomal site. The term “mutagenesis” denotes a chemical or physical treatment that changes nucleotides in the genome of an organism. An example of a chemical mutagenesis is N-ethyl-N-nitrosurea (ENU) mutagenesis.

The term “exon” encompasses a segment of a gene which is decoded to give a mRNA product or a mature RNA product. Individual exons may contain protein coding DNA and/or noncoding DNA. The term “intron” denotes noncoding DNA which separates neighboring exons in a gene. During gene expression introns, like exons are transcribed into RNA but the transcribed intron sequences are subsequently removed by RNA splicing and are not present in mRNA. The term “regulatory region” relates to the nucleotide sequence which comprises regions which are necessary for the regulation of the transcription of the gene. These regions comprise e.g. promoters and enhancers and they can be located in 5′ region of the exons or in introns. The term “splicing site” encompasses the nucleotides at a) the end of an exon and the beginning of the intron and at b) the end of the intron and beginning of the exon that are required for the joining of two exons and removal of an intron during the processing of a primary transcript to a functional mRNA.

The term “cancer” refers to a neoplastic disease. Cancer cells unlike benign tumor cells exhibit the properties of invasion and metastasis and are highly dedifferentiated. The term “pulmonary emphysema” denotes a condition of the lung characterized by a size increase beyond normal of air spaces distal to the terminal bronchioles, caused either by dilatation of alveoli or by destruction of their walls. The term “cardiomyopathy” designates a primary noninflammatory disease of the heart muscle which is not a result of ischemic, hypertensive, congenital, valvular or pericardial disease.

The term “embryonic stem cell” denotes a cell line derived from undifferentiated, totipotent cells from the embryo.

The term “selecting an agent for treating a symptom” encompasses choosing a composition for management of the condition.

The term “symptom” relates to subjective evidence of disease in an individual's condition.

The term “applying one or more agents” relates to administering one or more compositions. The compositions can be administered orally, by inhalation, parenterally, e.g. intravenously, subcutaneously, intraperitoneally or intramuscularly, or topically, e.g. ophtalmically, vaginally, rectally or intranasally.

The term “differential LTBP-4 gene or protein expression or expression level” denotes transcription or translation of LTBP-4 gene, which is reduced or increased. Preferably, there is a lack of transcription or translation.

The term “defect in the LTBP-4 gene” relates to a mutation, i.e. a nucleotide change in the LTBP-4 gene.

The term “LTBP-4 gene allele status” denotes the state of the DNA sequence in both alleles of LTBP-4, i.e. whether the LTBP-4 gene contains a mutation and whether this mutation is heterozygous or homozygous .

The term “RT-PCR” encompasses a two-step protocol for synthesizing cDNA molecules, wherein cDNA strands are synthesized by reverse transcriptase, mRNA as a template, and the specific cDNA strand is amplified by PCR. The term “Northern analysis” relates to a molecular hybridization method in which the target consists of RNA molecules that have been size fractionated by gel electrophoresis and subsequently transferred to a membrane. The term “microarray analysis” refers to large scale expression analyses where tens of thousands of mRNAs from an individual are immobilized on a solid surface, preferably glass, and are hybridized to a LTBP-4 specific probe. The term “antibody directed to LTBP-4 protein” denotes a protein that is synthesized by a B lymphocyte and recognizes specific epitopes on a LTBP-4 protein.

The term “mutation screening” relates to searching for nucleotide changes in the DNA or cDNA of an individual. This can be carried out e.g. by sequencing.

The term “means to detect the LTBP-4 gene or protein expression or expression level or LTBP-4 gene allele status” denotes resources to characterize whether LTBP-4 gene is transcribed or LTBP-4 protein is translated and whether the level of transcription/translation is normal, decreased or increased or whether LTBP-4 gene carries a nucleotide change. The resources comprise antibodies directed to LTBP-4 protein, a probe, which hybridizes to LTBP-4 gene and primer pairs which are suitable to amplify regions of LTBP4-gene or LTBP-4 cDNA.

In the present invention a strong hypomorphic allele of the mouse LTBP-4 gene was generated by gene trap mutagenesis in embryonic stem cells. Mice homozygous for the gene trap integration expressed only trace amounts of LTBP-4 and developed severe pulmonary emphysema, cardiomyopathy and colorectal cancer. The abnormalities were caused by a defect in the assembly of elastic fibers and by a defective TGF-β transport to the extracellular space.

Interestingly, the hypomorphic allele was generated by an inverse orientation exon trap insertion into an intron of the LTBP-4 gene, which theoretically should not interfere with gene expression. However, intron insertions have been shown previously to cause mutations. Examples include the ob2J mutation induced by a retrotransposon insertion into the first intron of the mouse leptin gene (Moon, B. C. & Friedman, J. M. (1997) Genomics, 42, 152-156), the mdr-3 mutation induced by a retroviral LTR insertion into the 22^{nd} intron of the mouse multi-drug resistance gene-3 (Jun, K., et al. (2000) Mamm Genome 11, 843-848), and the Mbp mutation induced by a retrotransposon insertion into the 3^{rd} intron of the rat myelin basic protein gene (O'Connor, L. T. et al. (1999) J Neurosci 19, 3404-3413). In each case, aberrant splicing precluded the expression of a fully processed, full length transcript. In cells of the present invention multiple atypical LTBP-4 transcripts were detected by RT-PCR, indicating that the mutation was caused by aberrant splicing.

The disease model of the present invention demonstrates a dual function of the TGF-β binding proteins. While the absence of elastic fibers in the lung and colon underscores the structural requirement of LTBP-4, the lack of extracellular TGF-β implicates LTBP-4 in TGF-β signaling. As TGF-β inhibits epithelial cell proliferation, particularly in the colon, it can be concluded that its absence from the colonic ECM is the most likely oncogenic trigger for the development of colon cancer in mice of the present invention. Indeed, several studies have associated defects in TGF-β signaling with colorectal cancer. For example, mice with null mutations in the TGF-β signal transducing protein-Smad 3, develop tumors which are similar to the tumors growing in 3C7 mice (Zhu, Y., et al. (1998) Cell 94, 703-714). Furthermore, mutations in the TGF-β signal transducing proteins Smad 2 and Smad 4 or mutations in the TGF-β II receptors are very common in human colorectal cancers, suggesting that TGF-β and its downstream targets have tumor suppressor functions (Markowitz, S. et al. (1995) Science 268, 1336-1338), Riggins, G. J. et al. 1997) Cancer Res 57, 2578-2580, Zhou, S. et al. (1998) Proc Natl Acad Sci USA 95, 2412-2416). Thus, the present invention demonstrates that LTBP-4 is important for the integrity of the ECM and prevents oncogenic transformation, cancer cell invasion and metastatic spread.

One of the most advantageous observations made in this invention is the tissue selectivity of the LTBP-4 mutation. Only the lung, heart and colon developed disease in mutant mice of the invention, indicating that LTBP-4 is a highly tissue specific protein. Moreover, this tissue specificity also shows that despite the extensive structural similarities, latent TGF-β binding proteins have little if any overlapping functions. Accordingly, the mutant mice express normal levels of LTBP-1 and LTBP-2, demonstrating that these isoforms cannot substitute for LTBP-4 (FIG. 3C). In addition, gene knock out studies have assigned LTBP-2 and LTBP-3 to completely different functions. For example, mice expressing null alleles of LTBP-2 die very early in development, most likely due to defective implantation (Shipley, J. M. et al. (2000) Mol Cell Biol 20, 4879-4887). In contrast, LTBP-3 knock out mice develop extensive skeletal abnormalities due to defective bone formation.

In conclusion, the present invention provides an animal model of human disease that reveals some crucial functions of LTBP-4. In view of these functions, the animal model of the invention can be used to provide novel treatments for connective tissue disorders, pulmonary emphysema, cardiomyopathy and cancer, and to obtain information about cancer and about the normal and defected function of connective tissue, lung and heart. Furthermore, the animal model of the present invention can be used for the dissection of the molecular mechanisms of LTBP-4 and the TGF-β pathway and for the identification and cloning of modifier genes able to modify, aggravate, reduce or inhibit the phenotype associated with cancer, pulmonary emphysema and/or cardiomyopathy or other conditions occurring in the animal model of the invention. Moreover, the animal model can be used for identification of early diagnostic markers for cancer and/or pulmonary emphysema and/or cardiomyopathy or other conditions occurring in the animal model of the invention. In addition, the animal model of the present invention can be used for the monitoring of the activity of agents useful in the prevention or treatment of cancer and/or pulmonary emphysema and/or cardiomyopathy or other conditions occurring in the animal model of the invention and as a test model system for agents suspected of promoting or aggravating cancer and/or pulmonary emphysema and/or cardiomyopathy or other conditions occurring in the animal model of the invention.

### DESCRIPTION OF FIGURES

The present invention is further illustrated by the following figures:

FIG. 1: Colorectal cancer in mice of the present invention (the mice of the present invention designated herein as 3C7 mice). A. Rectal prolapse (left panel) and digestive tract preparation with colorectal tumor (arrow) (right panel) of a 4 month old mouse. B. Histological sections through the colorectal region of wild type (+/+) and homozygous 3C7 mice (−/−). Top: PAS staining at ×200 magnification. Bottom: Immunostaining for the proliferation associated antigen Ki-67. Arrow points to an aberrant, dysplastic crypt. The −/− phenotype was stable for over 6 generations of backcrossings to a C57BL/6 genetic background.

FIG. 2: Pulmonary emphysema and cardiomyopathy in 3C7 mice. A. Heart/lung preparations from wild type (+/+) and homozygous 3C7 (−/−) mice. B. Lungs. Hematoxylin/eosin stain at ×200 magnification. C. Hearts. Hematoxylin/eosin stain at ×200 magnification. The −/− phenotype was stable for over 6 generations of backcrossings to a C57BL/6 genetic background.

FIG. 3: Gene trap disruption of the LTBP-4 gene locus. A. Predicted intron/exon structure of the mouse LTBP-4 gene with the gene trap integration site. The exon/intron structure was compiled by using the full length mouse cDNA sequence (SEQ ID NO:1, SEQ ID NO:2), the full length human cDNA sequence, and the available genomic sequence of mouse chromosome 7 synthetic to human chromosome 11. B. Structure of the U3Cre gene trap provirus (top) and Northern blot analysis of 3C7 ES cells (bottom). Polyadenylated mRNAs from differentiating ES cells were blotted onto nylon filters and hybridized to ³²P labeled Cre- or L32-specific probes. C. LTBP-4 expression in wild type (+/+) and homozygous 3C7 (−/−) mice. Top: Northern blot analysis. Polyadenylated RNAs from selected mouse tissues were blotted onto nylon filters and hybridized to a ³²P labeled LTBP-4 probe. The probe was a 505 kb fragment extending between nucleotides 862-1367 of the LTBPS-4 cDNA (SEQ ID NO:2) (for location see A.). Bottom: Immunoblotting with human anti-LTBP-4 and anti-LTBP-1 antibodies. Proteins from selected tissue digests were separated on denaturing polyacrylamide gels, transferred to nitrocellulose membranes and reacted with polyclonal anti-LTBP-1 or anti-LTBP-4 antibodies.

FIG. 4: Sequence of intron 5 of the mouse LTBP-4 gene (SEQ ID NO: 5). An arrow indicates the gene trap integration site. Splice donor and splice acceptor sites have been underlined.

FIG. 5: Elastic fibers in selected tissues of homozygous 3C7 mice. Sections were stained with Weigert's resorcin-fuchsin and analyzed at ×200 magnification. (+/+wild type mice; −/−3C7 mice).

FIG. 6: Intracellular versus extracellular TGF β 1 in selected mouse tissues. Tissue sections were stained with polyclonal TGF-β antibodies specific for the intracellular (LC) or extracellular (CC) form of TGF-β 1.

### EXAMPLES

The invention is exemplified by the following illustrative but non-limiting examples:

### Example 1

### A Stem Cell Clone Recovered from a Gene Trap Screen Generates a Complex Disease Phenotype in Transgenic Mice

From a larger screen for gene trap integrations into genes induced during ES cell differentiation (Thorey, I. S. et al. (1998) Mol Cell Biol 18, 3081-3088), one integration was obtained (designated herein as 3C7) that generated a complex disease phenotype when bred to homozygosity in transgenic mice.

129/Sv strain derived (D3) ES cells were grown on irradiated (32 Gy) MEF feeder layers in DMEM supplemented with 15% (v/v) preselected and heat inactivated fetal calf serum (FCS) (Linaris, Bettingen, Germany), 100 mM nonessential amino acids (Gibco), 0.1 mM mercaptoethanol (Sigma), 1000 U/ml of leukemia inhibitory factor (LIF) (Esgro^{R}; Gibco/BRL), and 5 mg/ml penicillin and streptomycin. Differentiation was induced by withdrawing LIF and feeder layers for 4 days (Thorey, I. S. et al. (1998) Mol Cell Biol 18, 3081-3088). Infection of ES cells with the U3Cre gene trap virus was performed by incubating for 2 hours 150 ES cells with 50 β l of virus containing supernatant described previously (Thorey, I. S. et al (1998) Mol Cell Biol 18, 3081-3088).

129/Sv (D3) ES cell derived chimeras were generated by injecting C57BL/6 blastocysts. The resulting male chimeras were bred to C57BL/6 females and agouti offspring were tested for transgene transmission by tail blotting. Mouse tail DNA was cleaved with BgIII which does not cut provirus. The DNA was fractionated on 1% agarose gels, blotted onto Hybond N nylon filters (Amersham/Pharmacia, Piscataway, N.J.) and hybridized to a ³²P labeled provirus flanking sequence probe. Animals heterozygous for the gene trap insertion were backcrossed to C57BL/6 mice for at least six generations before analyzing the phenotypes in heterozygous and homozygous offspring.

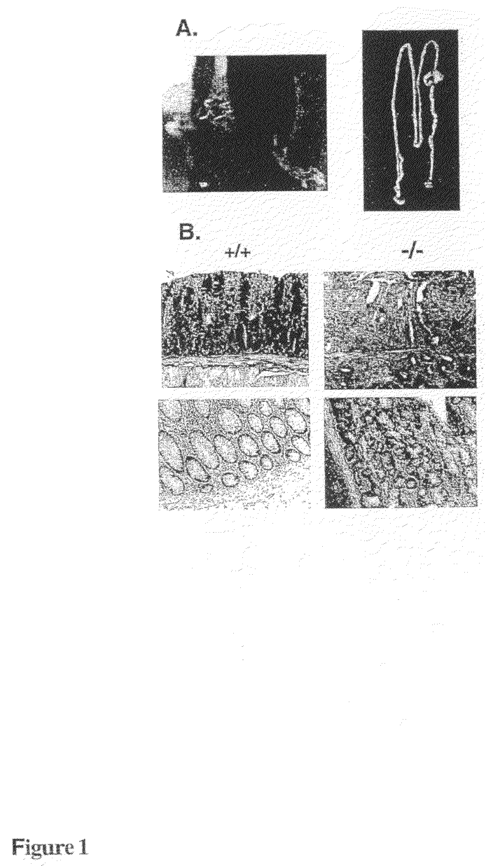

By the age of four weeks, 3C7 mice developed a rectal prolapse induced by an extensive edema in the mucosal layer of the colorectal region (FIG. 1A, left panel). Paraffin sections of mouse tissues were prepared and stained according to standard histopathology procedures known to the person skilled in the art. Microscopically, the region exhibited dysplastic and/or collapsed crypts with an increased number of goblet cells (data not shown). Lesions of this type are consistent with a preneoplastic stage of colon cancer and, indeed, by the age of 4-6 months, 3C7 mice developed visible tumors with diameters of 0.7-1.5 cm (FIG. 1A, right panel). Histologically, the tumors consisted of irregular crypts formed by highly proliferative anaplastic cells that infiltrated the mucosal and smooth muscle layers of the colonic wall (FIG. 1B). This pathology is typical for aggressive adenocarcinomas and is similar to the carcinomas developing in Smad 3 knock-out mice (Zhu, Y., et al. (1998) Cell 94, 703-714). As Smad 3 is a downstream target of TGF-β, the similarity between the tumors suggested that the tumors in both Smad 3 knock out and 3C7 mice developed as a consequence of defective TGF-β signaling.

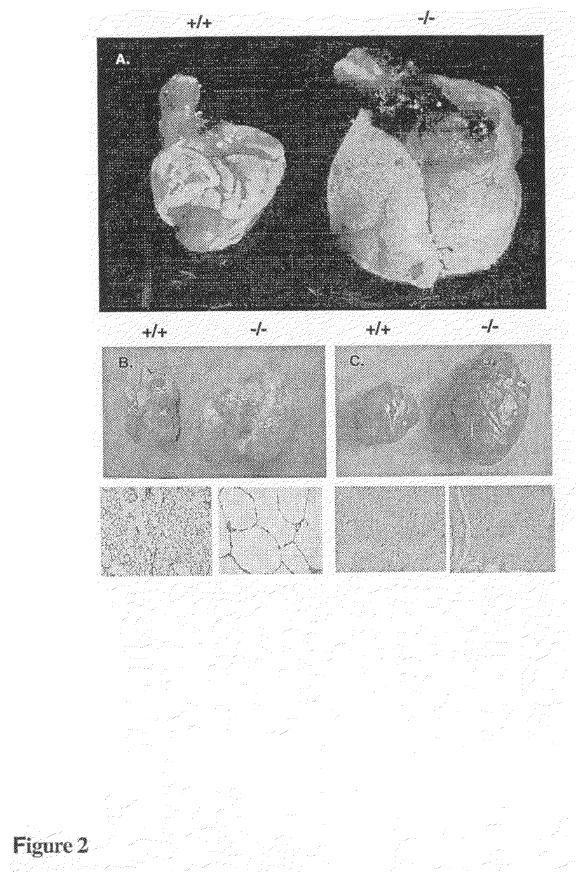

In addition to the tumors, 3C7 mice developed a pulmonary emphysema which, unlike the tumors, was already present at birth. The emphysema worsened with time such that by age of 4-6 months the lungs of 3C7 mice exceeded normal size by 4 fold (FIG. 2A, B). Histologically, alveolar spaces were enlarged, inflated and significantly reduced in number. They were surrounded by thin, dysplastic and frequently incomplete septal walls (FIG. 2B). The lobular connective tissue was diminished and replaced by mutifocal atelectatic areas. This resulted in an almost complete loss of pulmonary elasticity. The emphysema was associated with a cardiomegaly that involved biventricular dilation and myofibrillar hypertrophy (FIG. 2C). In most cases the cardiac/body weight index exceeded the normal value by 30%.

Clinically, animals were healthy up to 4-6 months of age but thereafter their general condition deteriorated. At this stage, mice were sacrificed due to severe dyspnea and prolapse infections that frequently progressed towards ulcerative proctitis. Autopsy revealed a massive inflammatory infiltration of the colonic wall that was associated with reactive splenomegaly. All other organs and tissues were microscopically normal based on analysis of liver, kidney, intestine, lymph nodes, brain, bone marrow, thymus, pituitary, thyroid, pancreas, ovaries, uterus, and testicles.

As heterozygous mice were indistinguishable from wild type mice and transgene inheritance was consistent with a Mendelian pattern, it was concluded that the gene trap had disrupted the function of an autosomal recessive gene.

### Example 2

### The Gene Trap Integration in 3C7 Mice is Located in the 5th Intron of the LTBP-4 Gene

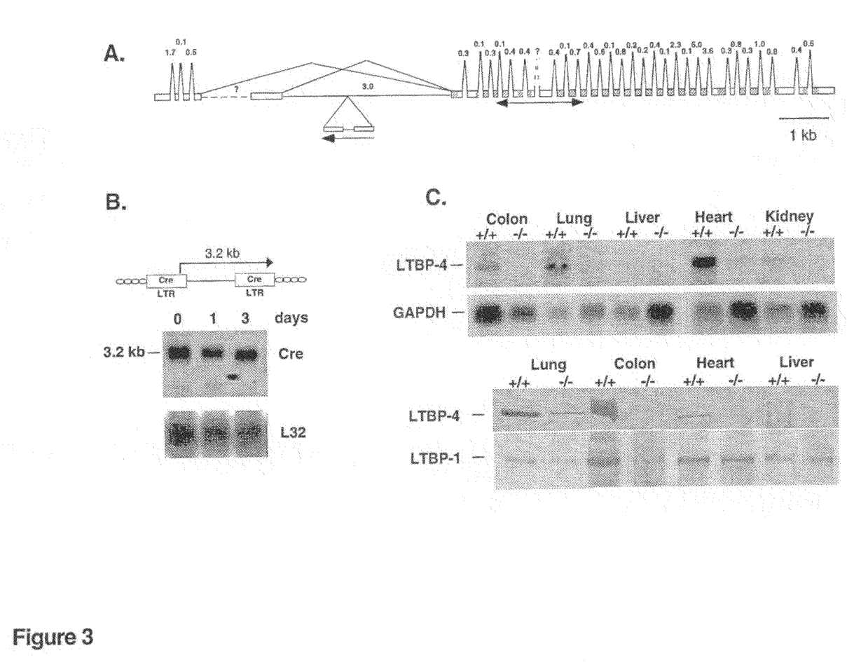

To identify the gene responsible for the 3C7 phenotype, 5′RACE was used to isolate cellular sequence co-transcribed with the provirus (gene trap sequence tag, GTST) (Wiles, M. V. et al. (2000) Nat Genet 24, 13-14). Normally, the type of retroviral gene trap employed in these experiments selects for integrations into exons (exon trap) and in most cases sequences upstream of the integration site are transcribed as cell-provirus fusion transcripts which can be easily identified by RT-PCR (Wempe, F. et al. (2001) Genome Biol 2, research0023.0021-002310). Since 5′RACE and Northern blotting showed no cell provirus fusion transcripts (FIG. 3B) it was concluded that in this case the gene trap, which is normally transcribed from an upstream cellular promoter, was activated instead by a nearby cellular enhancer. Enhancer activations with this type of gene trap have been observed, as it still contains a minimal promoter in the 5′LTR (FIG. 3B) (Thorey, I. S. et al. (1998) Mol Cell Biol 18, 3081-3088). Taken together, these results showed that the gene trap had integrated into an intron and was therefore inaccessible to cloning by RT-PCR. Inverse PCR and genome walking was used to isolate the genomic sequence spanning the provirus integration site.

Inverse PCR from genomic DNAs was performed using the blunt end restriction enzyme SspI and Cre-specific primers (Wempe, F., et al. (2001) Genome Biol 2, research0023.0021-002310). A 210 nt amplification product was cloned into the p-GEMT-vector (Promega, Madison, Wis.) and sequenced using an ABI 310 Genetic Analyzer (Perkin-Elmer, Foster, Calif.). The upstream provirus flanking sequence was extended by genome walking using the mouse GenomeWalker kit (Clontech, Palo Alto, Calif.) according to the manufacturer's instructions and the specific flanking sequence forward primers 5′-AAGAGGGAGAAGAAACTGGGCTGG-3′ (SEQ ID NO:3) and 5′-TCCTGTAGTGTTTGCCCCTG-3′ (SEQ ID NO:4). A 781 by genomic contig spanning the proviral integration site was amplified from the kit's EcoRV library and used as a probe to screen the lambda-TripleEx 5<<-Stretch PLUS mouse kidney cDNA library (Clontech, Palo Alto, Calif.) and the lambda EMBL3 mouse liver genomic library (Clontech, Palo Alto, Calif.). Positive plaques were purified by standard methodology and inserts were sequenced after subcloning into Bluescript vectors.

The 781 nt contig was cloned and sequenced and used to find matches in the public data bases. Cellular sequences adjacent to proviral integrations (gene trap sequence tags; GTSTs) were searched in the NCBI/NIH genomic databases (world wide website ncbi.nom.nit) using the Blast algorithm. As no matches were found after extensive searching, it was concluded that the trapped gene is still unknown. To identify the gene, Cdna- and genomic libraries were screened with the genomic contig with methods known to the person skilled in the art, thus resulting in the isolation of a 12 kb genomic fragment with two short stretches of homology to the human cDNA encoding the latent transforming growth factor beta binding protein 4 (LTBP-4; Genbank Acc. #AF051344; ref NM/003573.1; AF051345). The matching segments corresponded to exons 6 and 7 encoding the 5′ end of the first EGF-repeat, which is shared among known splice variants (Saharinen, J. et al. (1998) J Biol Chem 273, 18459-18469). (FIG. 3A). The splice variants LTBP-4L (long splice variant) and LTBP-4S (short splice variant) of the mouse LTBP-4 cDNA are shown in SEQ ID NO:1 and SEQ ID NO: 2, respectively.

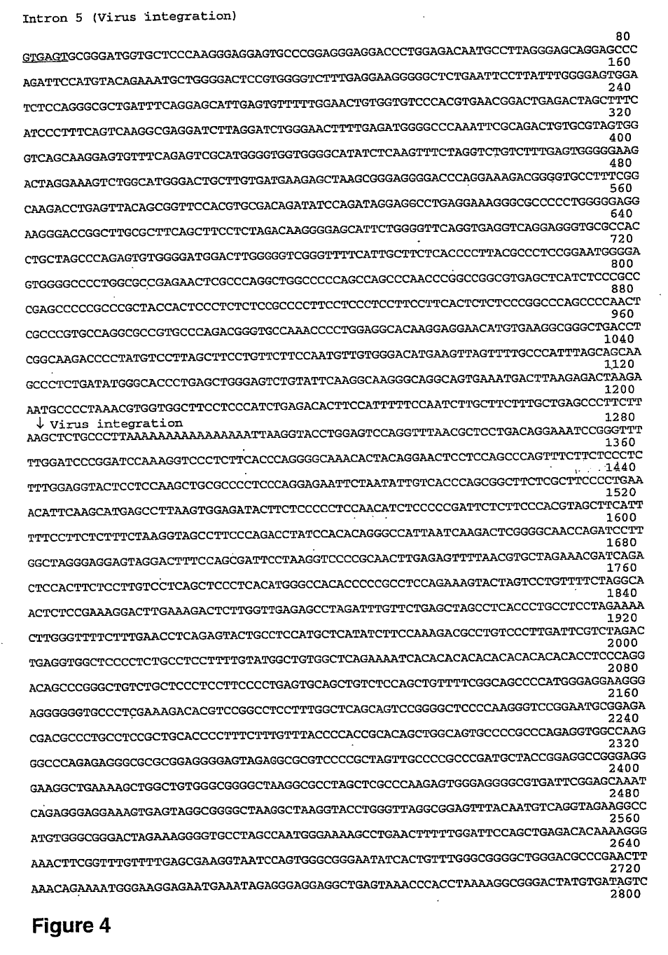

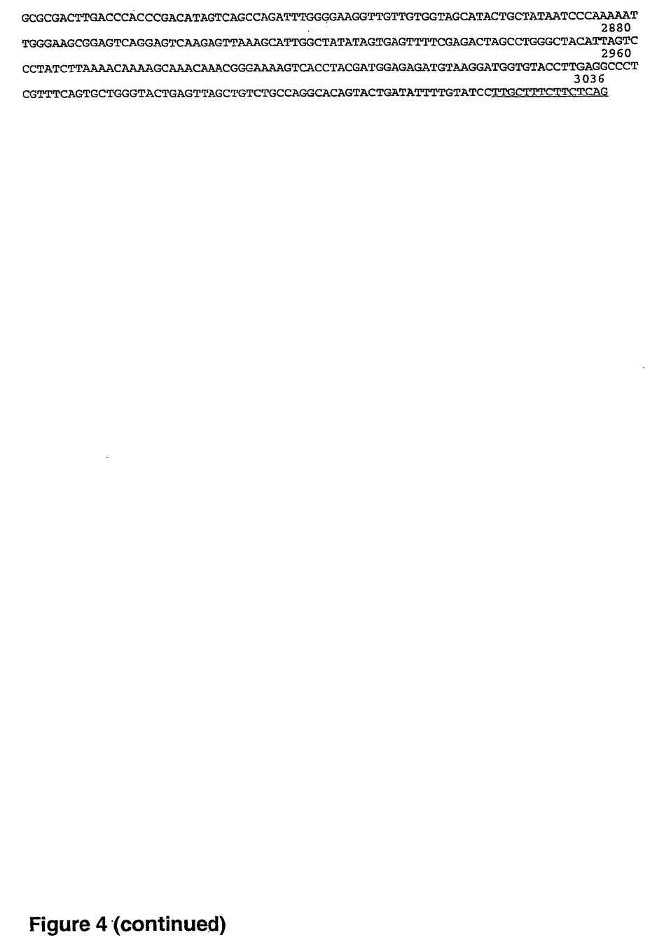

FIG. 3A shows that the gene trap integration had occurred into the 5^{th} intron of the mouse LTBP-4 gene in a reverse transcriptional orientation relative to the gene. This inverted orientation explains the absence of a cell-provirus fusion transcript and supports a gene trap activation by a cellular enhancer. The sequence of intron 5 of the mouse LTBP-4 which contains the gene trap integration is shown in FIG. 4 and SEQ ID NO: 5.

### Example 3

### The Proviral Integration in LTBP4 Severely Interrupts Gene Expression

To investigate whether the proviral intron disruption interfered with gene expression, mRNA from a variety of mouse tissues was hybridized on Northern blots to an LTBP4 specific probe. For Northern blots 2.5 □g of poly (A)⁺ mRNAs purified with the FastTrack 2.0 mRNA purification kit (Invitrogen, Carlsbad, Calif.) were fractionated in 1% formaldehyde-agarose gels, transferred onto Hybond N nylon filters and hybridized to a ³²P-dCTP-labeled LTBP-4 specific probe. The probe was a 505 by fragment located between nucleotides 862-1367 in the mouse LTBP-4S cDNA (SEQ ID NO:2). All probes were prepared by random priming using the Rediprime Amersham/Pharmacia, Piscataway, US) kit. Blots were exposed to Kodak-Biomax autoradiography film. As shown in FIG. 3C (top), mice homozygous for the gene trap integration expressed less than 2% of normal LTBP-4 mRNA. Transcript levels in wild type mice were significantly higher in the lung, heart and colon than in other tissues, demonstrating a link between the phenotype and LTBP-4 expression (FIG. 3C).

To test whether protein levels were similarly decreased, the same range of tissues was analyzed by immunoblotting using an anti-LTBP-4 specific antibody (Saharinen, J., et al. (1998) J Biol Chem 273, 18459-18469). Equal amounts of selected tissues were homogenized in ice-cold RIPA buffer (150 mM NaCl, 1% NP-40, 0.5% sodium deoxycholate in 50 mM Tris-HCl, pH 7.2). Following centrifugation, the deoxycholate insoluble pellet was washed in PBS and digested with plasmin (Taipale et al. (1992) J. Biol. Chem. 267: 25378-25384). After adding protease inhibitors (Roche Diagnostics, Mannheim, Germany), the supernatants were concentrated in Microcon 30 concentrators (Amicon, Millipore Corp., Bedford, Mass.). Proteins were fractionated on 7% SDS/PAGE gels and transferred to Protran nitrocellulose membranes (Schleicher & Schuell, Dassel, Germany). Immunodetection after treatment with polyclonal anti-LTBP-1 or anti-LTBP-4 antibodies was performed (Saharinen et al. (1998) J Biol Chem 273, 18459-18469). As shown in FIG. 3 C (bottom), protein was hardly detectable in 3C7 mice and levels closely followed the pattern of mRNA expression in the wild type mice. Taken together, the results showed that the gene trap integration into the 5^{th} intron of the LTBP-4 gene resulted in a nearly null allele.

### Example 4

### Elastic Fibers in the Lung and Colon Require LTBP-4

Together with fibrillins, LTBPs form a sheath of microfibrills that surrounds the elastic fiber's elastin core. Fibrillins are essential for the integrity of elastic fibers and mutations in the fibrillin genes are common in connective tissue disorders such as Marfan's syndrome. Since it was shown that 3C7 mice fail to assemble elastic fibers, particularly in the lung and colon, the animal of the present invention is a suitable model to investigate the role of LTBP-4 for the assembly of elastic fibers. To test this, elastic fibers were visualized using an elastin-specific histological stain. Tissues were fixed in 4% buffered formalin, processed through a graded series of alcohol, cleared in xylene and placed in a tissue processor for 8 hours. 5 micron paraffin sections were prepared with a rotary microtome, placed on lysine coated glass slides. After incubating for 45 minutes at 65° C., slides were deparaffinized in xylene. After rehydrating in double distilled H₂O, slides were immersed in Weigert's resorcin-fuchsin solution (70% v/v ethanol, 1% v/v hydrochloric acid conc, and 0.2% v/v resorcin-fuchsin [Chroma, Munster, Germany]).) for 6 hours at room temperature. Following washing in ddH₂O slides were dehydrated in alcohol and mounted in permount.

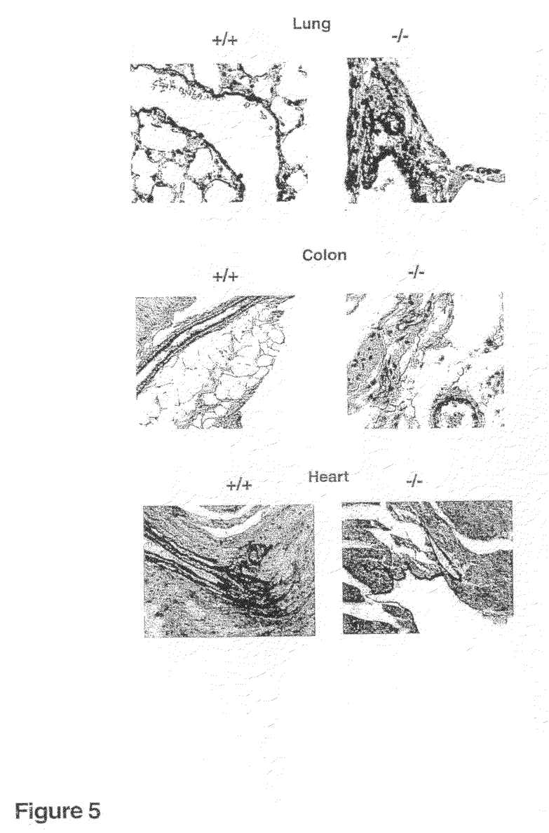

FIG. 5 shows that the lung and colon of 3C7 mice lacked elastic fibers and exhibited instead multiple patches of fragmented and condensed elastin (FIG. 5).

Surprisingly, elastic fibers in the heart appeared unaltered by the mutation, indicating that the severe cardiomyopathy is at least in part secondary to the emphysema (FIG. 5). Taken together, the results show that LTBP-4 is essential for the assembly of elastic fibers.

### Example 5

### Export of TGF-β1 to the ECM Requires LTBP-4

It has recently been shown that of all TGF-β isoforms, only TGF β1 binds LTBP-4 in vitro (Saharinen, J. & Keski-Oja, J. (2000). Mol Biol Cell 11, 2691-2704). Furthermore, LTBPs are responsible for the export of TGF-β to the ECM. To test if mice lacking LTBP-4 would also lack extracellular TGF β 1, anti-TGF.-β 1 antibodies were used that recognize TGF.-β 1 epitopes selectively displayed inside (LC) or outside (CC) the cell (Flanders, K. C. et al. (1989) J Cell Biol 108, 653-660, Thompson, N. L. et al. (1989) J Cell Biol 108, 661-669). For immunostainings, deparaffinized and rehydrated tissue slides were first treated for 30 minutes with 30% H₂O₂ to block the endogenous peroxidase. After rinsing in ddH₂O and soaking in PBS for 5-10 minutes, slides were treated with 10% w/v BSA in PBS to eliminate nonspecific protein binding sites. The slides were then exposed overnight at 4° C. to the polyclonal TGF.-β 1 anti-CC (1-30) and anti-LC (1-30) antibodies at concentrations of 1 ug/ml and 4 β g/ml, respectively (Flanders, K. C. et al. (1989) J Cell Biol 108, 653-660). After removing excess antibody, slides were treated with biotin-labeled anti-mouse (Dianova, Hamburg) antibody for 30 minutes at 37° C. and finally with horse-radish peroxidase (HRP) labeled streptavidine (Zymed, CA) for 20 minutes at 37° C. After washing, slides were incubated in diaminobenzidine (Sigma, St. Louis, Mo.) for 10 minutes at room temperature. Slides were examined under a conventional microscope after removing the excess substrate in ddH₂O.

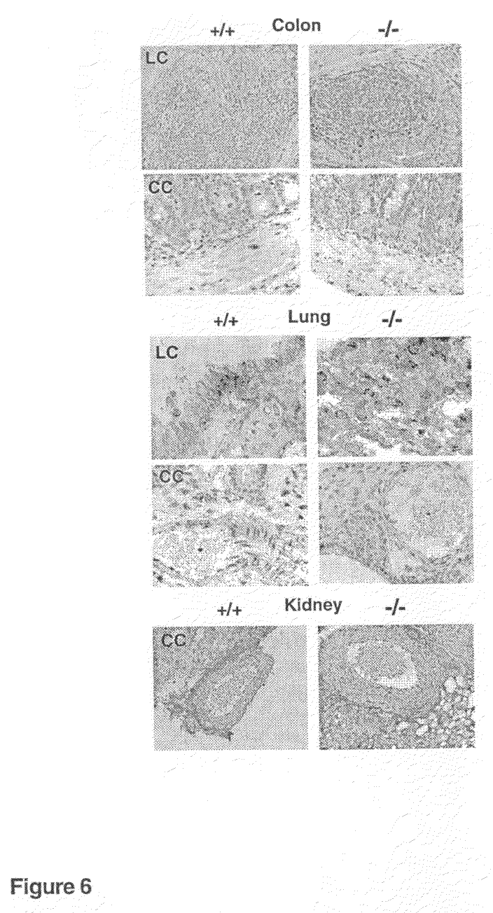

Immunohistochemical analysis showed that in 3C7 mice the lung, colon and heart contained no extracellular TGF-β1. As intracellular levels were normal, the results demonstrate a defect in exporting TGF-β1 to the ECM. This defect is tissue specific since the kidneys of 3C7 mice had normal extracellular TGF-β1 levels (FIG. 6).

1-24. (canceled)

25. A transgenic mouse model for colorectal cancer, pulmonary emphysema or cardiomyopathy comprising a transgenic mouse that produces suboptimal levels of latent transforming growth factor β binding protein 4 (LTBP-4); wherein the transgenic mouse comprises a mutation resulting in homozygous disruption of both endogenous alleles of a gene encoding LTBP-4, and the transgenic mouse develops colorectal cancer, pulmonary emphysema or cardiomyopathy.

26. The mouse model of claim 25, wherein said mutation is an insertion, deletion or a substitution mutation.

27. The mouse model of claim 25, wherein said mutation is generated by gene targeting, gene trap integration or mutagenesis.

28. The mouse model of claim 25, wherein the mutation has occurred in an exon, intron, regulatory region or splicing site of the LTBP-4 gene.

29. The mouse model of claim 28, wherein the mutation is in intron 5 of the LTBP-4 gene.

30. A cell isolated from a transgenic mouse whose genome comprises a homozygous disruption of both endogenous alleles of the LTBP-4 gene such that said gene produces suboptimal levels of latent transforming growth factor β binding protein 4 (LTBP-4), and the transgenic mouse develops colorectal cancer, pulmonary emphysema or cardiomyopathy.

31. The cell of claim 30, wherein the cell is from colon, lung or heart of the transgenic mouse.

32. An isolated, transgenic mouse cell whose genome comprises a homozygous disruption of both endogenous alleles of the LTBP-4 gene, wherein the LTBP-4 gene includes a gene trap integration mutation in intron 5 of the LTBP-4 gene.

33. The cell of claim 32, wherein the cell is an embryonic stem cell.

34. A method for selecting an agent for treating colorectal cancer, pulmonary emphysema or cardiomyopathy comprising:
a) applying one or more agents to be tested to the mouse model of claim 25, and
b) determining whether the colorectal cancer, pulmonary emphysema or cardiomyopathy occurring in the mouse model has changed as a result of application of said one or more agents.

35. A transgenic mouse comprising a gene trap mutation in intron 5 of both alleles of a gene encoding latent transforming growth factor β binding protein 4 (LTBP-4), wherein the mouse produces suboptimal levels of LTBP-4, and develops colorectal cancer, pulmonary emphysema or cardiomyopathy.

36. A method for selecting an agent for treating colorectal cancer, pulmonary emphysema or cardiomyopathy comprising:
a) applying one or more agents to be tested to the transgenic mouse of claim 35, and
b) determining whether the colorectal cancer, pulmonary emphysema or cardiomyopathy occurring in the mouse has changed as a result of application of said one or more agents.

37. An isolated cell from the transgenic mouse of claim 35.

38. The isolated cell of claim 37, wherein the cell is a colon cell, a lung cell, or a heart cell.

39. The isolated cell of claim 37, wherein the cell is an embryonic stem cell.
